(19) United States
(12) Patent Application Publication
(10) Pub. No.: US 2010/0010333 A1
(43) Pub. Date: Jan. 14, 2010

(54) Bipolar, Non-Vectorial Electrocardiography

(76) Inventor: Jorge Hernando Ordonez-Smith, Columbia, MD (US)

Correspondence Address: JORGE HERNANDO ORDONEZ-SMITH, 5955 CAMELBACK LANE, COLUMBIA, MD, 21045, US

(21) Appl. No.: 12/554,979

(22) Filed: Sep. 7, 2009

Related U.S. Application Data
(63) Continuation-in-part of application No. 11/193,140, filed on Jul. 29, 2005.
Application No. 11/193,140 is a continuation-in-part of application No. 11/163,140, filed on Oct. 6, 2005, now abandoned.

(51) Int. Cl. A61B 5/0402

(52) U.S. Cl. 600/372; 600/509

(57) ABSTRACT

An improved method for registering the changes in electrical potentials present on the surface of the body in association with the contraction of the heart by recognizing that today's art hypothesis on the genesis of such potentials are unsustainable. The new “Bipolar non-Vectorial Leads” are obtained by paring a distal “Common or Positive Electrode” placed on the left leg with an “Exploring or Negative Electrode” placed near the myocardium on areas where the electrical potentials generated by the different structures of the myocardium are prevalent. The approximate twelve leads will sample all the areas were each myocardial structure is prevalent. The leads so obtained are to be analyzed as generated on the surface of the myocardium and conducted throughout through the body to the entire surface by the muscular masses that are in close contact with the different structures of the myocardium. The final report besides the printed electrocardiographic traces includes all the digital data sets, obtained by the electrocardiograph, saved on a digital disk.

### CROSS-REFERENCE TO RELATED APPLICATIONS

This application is a continuation-in-part to the application Ser. No. 11/163,140 filled on Oct. 6, 2005.

### OTHER REFERENCES

Einthoven, W.: Le Telecardiograme. Arch. Intern. Physiol. 1906; 4: 132-164

Einthoven, W.: The Different Forms of the Human Electrocardiogram and Their Signification, Lancet, 1912; I: 853-861

Einthoven, W., Fahr, G., de Waart, A.: On the Direction and Manifest Size of the Variations of Potential in the Human Heart and on the Influence of the Position of the Heart on the Form of the Electrocardiogram, Pflüger's Arch. F. Physiol., 1913; 150: 275-315

Goldberger, E.: A Simple, Indifferent, Electrocardiographic Electrode of Zero Potential and a Technique of Obtaining Augmented, Unipolar, Extremity Leads, Am. Heart', 1942; 23: 483-492

Katz, L. N., and Korey, H.: The Manner in Which the Electric Currents Generated by the Heart Are Conducted Away. Am. J. Physiol. 1935; 111: 83-90

Lewis, T.: Interpretations of the Initial Phases of the Electrocardiogram with Special Reference to the Theory of “Limited Potential Differences”, Arch. Int. Med., 1922; 30: 269285

Ordóñez-Smith, J. H.: Study on the theories of: “Einthoven's Equilateral Triangle”, “Wilson's Central Terminal” and the “Unipolar Leads of Goldberg and Wilson”, Rev. Col. Cardiol., 2000; 8: 139-150

Ordóñez-Smith, J. H.: Morfología del electrocardiograma: Una nueva teoria, Medicina 2008; 30 (80): 8-26

Supplementary Report by the Committee of the American Heart Association for the Standardization of Precordial Leads, Am. Heart', 1938; 15: 235-239

Waller, A. D.: The Electromotive Properties of the Human Heart, Brit M. J., 1888; I: 751-754

Waller, A. D.: On the Electromotive Changes Connected with the Beat of the Mammalian Heart and of the Human Heart in Particular, Phil. Trans. Roy. Soc. B., 1889; 180: 169-194

Wilson, F. N., Johnston, F. D., Macleod, A. G., Barker, P. S.: Electrocardiograms That Represent the Potential Variations of a Single Electrode, Am. Heart', 1934; 9: 447-458

Wilson, F. N., Johnston, F. D., Rosenbaum, F. F., and Barker, P. S.: On Einthoven's Triangle, the Theory of Unipolar Electrocardiographic Leads, and the Interpretation of the Pericardial Electrocardiogram, A. Heart', 1946; 32: 277-310

### REFERENCE TO A MICROFICHE APPENDIX

Not applicable.

### STATEMENT REGARDING FEDERALLY SPONSORED RESEARCH

Not applicable

### BACKGROUND OF THE INVENTION

US patent Class: 600/5095 516, 517, 519, 523

IPC: A61 B/0402

1. Field of the Invention

The present invention relates to the acquisition and analysis of electrocardiographic recordings to facilitate the recognition of cardiac pathology and the understanding of the genesis of such anomalies. It is based in the discoveries that today's accepted hypothesis on which the genesis of the electrocardiographic traces depends to be valid are erroneous.

2. Description of the Related Art

Augustus Desire Waller (Waller, 1888 and 1889) did the first human electrocardiogram by immersing both hands of his assistant in containers of water and connecting them to a mercury electrometer. Initially, cardiograms were recorded using this technique. Only the hands, the feet, and the tongue were used to measure the differences in potential.

Later, Wilhelm Einthoven M.D. invented the string galvanometer (Einthoven, 1906) and was able to obtain more accurate recordings. Einthoven changed the terminology, established by A. D. Waller M. D. for the different deflections produced by the heart, in 1912 (Einthoven, 1912). Einihoven changed Waller's auricular deflection a to “P wave”, Waller's ventricular component V1 to “QRS complex”, and Waller's ventricular component V2 to “T wave”. Additionally he named a third ventricular component the “U wave”. This nomenclature is still in use today. Einthoven, Fahr, and de Waart (Einthoven et al, 1913) demonstrated the mathematical relationship, LIII=LII−LI, between the three standard leads and introduced the schema of the Equilateral Triangle to explain and calculate the changes that occur in the electrical axis of the heart. Sir Thomas Lewis' “Theory of Limited Potential Differences” (Lewis, 1922) strongly supported Einihoven's hypothesis by explaining how the different waves of the ORS Complex were generated.

After F. N. Wilson, F. D. Johnston, F. D. Macleod, and P. S. Barker (Wilson et al, 1934) published the technique of obtaining unipolar leads based on Einthoven's hypothesis; controversy surrounding the genesis of the electrocardiogram was virtually non existent. E. Goldberger (Goldberg, 1942) discovered, while recording Unipolar V Leads of the extremities, that by disconnecting the extremity that was going to be recorded, and eliminating the resistances from “Wilson's Central Terminal” the shape of the lead did not change, but the amplitude was greater. He called this leads Augmented Unipolar Leads, or aV Leads. When F. N. Wilson, F. D. Johnston, F. F. Rosembaum and P. S. Barker published their Theory of Unipolar Leads” (Wilson et al, 1946), the hypothesis of Einthoven's “Equilateral Triangle” with its “Central Dipole” became the standard genesis of the electrocardiogram. Wilson et al. stated that by joining the electrodes of the three extremities, through high resistances to form a common electrode, the electrical potential of this terminal was equal to or very near zero throughout the entire cardiac cycle. By coupling this Central Terminal to an exploring electrode placed in any area of the body, the electrocardiographic trace would show only the changes in potential occurring in that area of the body.

During the early years of electrocardiography, there was a lack of consensus as to which leads to utilize and what should be considered a normal electrocardiogram. The disagreement was due to the plethora of theories pertaining to how the changes in potential were produced and to the near infinite number of different recordings that are labeled normal. Consequently, medical societies of different countries (AMA, 1938) created a Standard of Electrocardiographic Leads which remains unchanged today.
Today's EKG consists of twelve leads: the three standard leads of Einthoven (Lead I, Lead II, and Lead III), described by Einthoven in 1912, the three Augmented Unipolar Leads of Goldberger (aVr, aVl, and aVf), described by Goldberger in 1942, and the six Unipolar Precordial Leads of Wilson (V1, V2, V3, V4, V5, and V6), described by Wilson et all in 1934. All the twelve leads use the distal electrodes R, L, and F. The precardial leads besides the three distal electrodes use a forth electrode, the exploring electrode, placed on the precardial area. These twelve leads are interpreted as generated by the dipole in the center of Einthoven's equilateral triangle”, the three standard extremity leads are interpreted as “Bipolar Vectorial Leads and the other nine leads are interpreted as “Unipolar Vectorial Leads”.

The analysis of the electrocardiographic trace is based on the absolute validity of the postulates of Einthoven's theory of the Equilateral Triangle with its Central Dipole, of Wilson's postulates for his Central Terminal of zero potential and the validity of Goldberger's terminals of zero potential.

Einthoven's postulates are:
a. “The human body is a flat homogeneous plate in the form of an equilateral triangle”,
b. “The heart is represented by a spot in the central point of the triangle”,
c. “Inside the spot two points represent the dipole that gives the direction of the maximal electrical potential of the heart at any given instant,”
d. “The distance between the central points is very small compared with the length of the side of the triangle.” (Einthoven, 1913; p 292-293)

The postulates of Wilson's Central Terminal of zero potential to obtain unipolar leads are:
a. “The sum of the differences in potential between any number of electrodes and a nodal point connected to these electrodes through equal resistances must be zero as a consequence of Kirchhoff's First Law.”
b. “The potential of the central terminal is equal at every instant to the mean of the potentials of the electrodes on the extremities.”
c. “the basis of the assumptions upon which the equilateral triangle of Einthoven, Fahr, and de Waart is based”,
d. “the assumption that the electrical forces of cardiac origin which are perpendicular to the plane of the standard limb leads have no significant effect upon the potential variations of the extremities” (Wilson et al, 1946; page 282).

Goldberger simply states that by disconnecting, from Wilson's Central terminal, the three resistances and the electrode of the limb to be investigated and pairing the other two electrodes, to form a modified central terminal, the potential of these new terminals is equal to zero, throughout the cardiac cycle, according to Kirchhoff's First Law also known as Kirchhoff's Junction Rule.

Personal Research

Through personal research, regarding Einthoven's theory of the Equilateral Triangle and its Central Dipole, I have found that:
a. Einthoven's Law, LIII=LII+LI, is valid because it fulfills the premises of the mathematical axiom,

If a−b=x, b−c=y, and c−a=z, then x+y+z=0 a)
and not due to the validity of Einthoven's Theory of the Equilateral Triangle and it's Central Dipole as is accepted in today's art (Ordóñez-Smith, 2000; page 154, 2008; page 9).
b. The human body is not a “flat, homogeneous plate in the form of an equilateral triangle” (Einthoven, 1913; page 282), as is accepted in today's art. It is cylindrical and is an inhomogeneous electrical conductor.
c. The heart does not propagate throughout the body the changes in electrical potential present on the surface of the body as a central dipole localized in a spot in the center of an equilateral triangle (Einthoven, 1913; pages 292-293), as is accepted in today's art. Instead, the monophasic potentials present on the surface of the heart are propagated though the muscular masses of the anterior and lateral walls of the chest and abdomen, the diaphragm, and the paraspinal musculature, that are in close contact with the different structures of the myocardium.
   In fact, electrical potentials generated by the contraction of the auricles are prevalent on:
      the supra and infra-clavicular areas of the right clavicle, and
      the lower left pre-sternal or left mammary areas.
   The changes of electrical potentials generated by the contraction of the right ventricle are prevalent on:
      the anterior surface of the cephalic two thirds of the right hemi-thorax.
   The electrical potentials generated by the contraction of the antero-lateral surface of the left ventricle are prevalent on:
      the anterior and lateral surfaces of the lower two thirds of the left hemi-thorax.
   The electrical potentials generated by the contraction of the postero-inferior surface of the left ventricle are prevalent along:
      the distal half of the posterior surface of the left hemi-thorax,
      the left lower back, and
      both legs (Ordóñez-Smith, 2008; page 20).
d. The distance between the surfaces of the different structures of the myocardium, were the electrical potentials exist, and the three extremities R, L, and F can not be considered “very small” (Einthoven, 1913; page 293), as is accepted in today's art. Those distances are different for each extremity and are close for both arms and can not be considered small (Ordóñez-Smith, 2008; page 11).

Regarding Wilson's assumptions over his Central Terminal of Zero Potential I have found that:
a. Wilson's statement, “The sum of the differences in potential between any number of electrodes and a nodal point connected to these electrodes through equal resistances must be zero as a consequence of Kirchhoff's First Law” (Wilson et al, 1946; page 282), as is accepted in today's art is flawed. Kirchhoff's First Law also known as Kirchhoff's Junction Rule deals with the flow of electrical current through an electrical junction within a closed electrical circuit, not with the electrical potential of the junction (Ordóñez-Smith, 2008; page 14).
b. Wilson's statement: “the potential of the central terminal is equal at every instant to the mean of the potentials of the electrodes on the extremities” (Wilson et al, 1946; page 282), as is accepted in today's art also is flawed. The potential of the central terminal, according to Kirchhoff's Second Law, also known as Kirchhoff's Loop Rule, is equal to the highest electrical potential of the electrical circuit loops connected to a junction within a closed electrical circuit (Ordóñez-Smith, 2008; page 14).
c. Einthoven's Law is valid because it fulfills the premises of a mathematical axiom (Ordóñez-Smith, 2000; page 154, 2008; page 9) and not to “the basis of the assumption upon which the equilateral triangle of Einthoven, Fahr, and de Waart is based” (Wilson et al, 1946; page 282), and as is accepted in today's art,
d. “The assumption that the electrical forces of cardiac origin which are perpendicular to the plane of the standard limb leads have no significant effect upon the potential variations of the extremities” (Wilson et al, 1946; page 282), that is also accepted by today's art, ignores the well known fact of the significant changes generated by myocardial infarcts of the anterior and posterior walls of the myocardium on the three standard leads and the lack of these changes by the lateral wall infarcts of the left ventricle that happen in the plane of the extremities (Ordóñez-Smith, 2008; pages 21-23).
Regarding Goldberger's three terminals of zero potential, I have found that:
a. They fall into Kirchhoff's Second Law, or Loop Rule, as does Wilson's Central Terminal. The electrical potential of the nodal point is equal to the highest potential of the two loops that form the terminal (Ordóñez-Smith, 2008; page 16), and not zero as described by Goldberger (Goldberger, 1942; page 486) and is accepted by today's art.

The so-called “Unipolar” leads are not “Unipolar”, they are complex “Bipolar” leads and do not represent the true changes in potential that are occurring at the sites where the exploring electrodes are placed, as is accepted in today's art. In reality, as long as all the electrodes are placed on or in the body, no true “Unipolar” leads can be recorded due to the fact that the changes in electrical potential generated by the contraction of the myocardium are present in and on the entire body and its surface and all are significant (Ordóñez-Smith, 2008; page 22).

From a mathematical point of view the three standard leads of the electrocardiogram are the first derivatives of three variable functions,

fR, fL, and fF. b)

These variable functions represent the changes of electrical potential generated by the monophasic potentials of the myocardium in each one of the different areas on the surface of the leg and both arms. In these variable functions the X-axis represents time in milli-seconds and the Y-axis represents electrical potential in milli-volts.

The morphogenesis of the different waves and segments of an electrocardiographic trace is due to the difference in amplitude, morphology and timing between the different monophasic electrical potentials generated by the contraction of the different structures of the myocardium and their conduction throughout the body by the muscular masses that are in close contact with them (Ordóñez-Smith, 2008; pages 21-23), and not the rotation of a dipole located in the center of an assumed Equilateral triangle, as is accepted in today's art.

### SUMMARY OF THE INVENTION

The invention is an improved method of recording and analyzing electrocardiographic leads based on the realization that Einthoven's Law is valid because it fulfills the mathematical axiom,

If a−b=x, b−c=y, and c−a=z, then x+y+z=0 a)

and not due to the validity of Einthoven's Equilateral Triangle. Placing three electrodes in any area of the body and recording an electrocardiogram will fulfill Einthoven's Law (Ordóñez-Smith, 2008; pages 9-10).

Today's accepted hypothesis about the genesis of electrocardiography: Lewis' Limited Potential Differences, Wilson's Central Terminal, and Goldberger's thre Central Terminals are totally dependant on the absolute validily of the hypothesis of Einthoven's Equilateral Triangle with the Central Dipole. Since Einthoven's theory is unsustainable in view of the new finding, every theory that depends on its absolute validity to be true becomes unsustainable too.

The leads and analysis of such leads in the new “Bipolar, Non-Vectorial Electrocardiography” are based in three facts:
1. The morphology of the different waves and segments of an electrocardiographic trace is not given by the changes in the rotation of Einthoven's Central Dipole, as is accepted in today's art. It is given by the differences in amplitude, morphology, and timing of the monophasic electrical potentials generated by the contraction of the different structures of the myocardium.
2. The monophasic potentials generated by the contraction of the heart do not propagate to the surface and throughout the body as if generated by a Central Dipole, as is accepted in today's art, but by the close contact of the different structures that comprise the heart with the musculature of the chest and abdominal walls, the diaphragm and the spine.
3. Any electrocardiographic lead obtained by placing the electrodes in or on the body is a bipolar lead.

To overcome the fact that the hypothesis, on which today's electrocardiography art is based, are unsustainable the new method records approximately twelve electrocardiographic leads by using approximately twelve identical individual amplifiers (instead of 8 amplifiers as in today's art), connecting the negative terminal of each amplifier to an “Exploring or Negative Electrode” and the positive terminal to a “Common or Positive Electrode” to obtain “Bipolar, Non-Vectorial Leads”.

The use of the same variable function (the changes in electrical potential on the left leg) in all the leads allows for the obtained information to be more evident and easier to analyze than the information that today's art traditional leads can supply. The values generated by the left leg are calculated and subtracted from all the leads. The final tracing will report the values generated by each of the “Exploring or Negative Electrodes” plus the values generated by the left leg. The digital data sets obtained by the recorder are to be saved on a “Digital Disc” that will be part of the permanent record. When subsequent electrocardiograms are recorded, the stored identified digital data sets from previous recordings are to be retrieved and compared by the recorder with the newly obtained identified digital data sets. The recorder will report any changes obtained by the recorder and report them together with the new electrocardiogram.

### OBJECTIVES AND FEATURES OF THE INVENTION

It is an objective of the present invention to obtain data that are more reliable and characteristic of the electrical potential differences generated by the contraction of the different structures of the myocardium in its normal and abnormal states.

It is a further objective of the present invention to provide a method of enhancing and facilitating the recognition of the changes of electrical potential differences on the body surface that are pathognomonic in the presence of myocardial pathology.

It is a further objective of the present invention to provide a method of analysis of the different changes of electrical potential on the surface of the body to facilitate the recognition of normal and abnormal patterns.

It is a feature of the present invention to acquire the changes of electrical potential on the surface of the body that occur in synchronization with the contraction of the heart at sites that are closer to the heart and in the areas where each different structure of the myocardium is prevalent.

It is a further feature of the present invention to analyze the changes of electrical potential on the surface of the body that occur in synchronization with the contraction of the heart as a result of characteristic conduction patterns of the monophasic electrical potentials generated by the different structures of the myocardium toward the body surface.

It is a further feature of the present invention to calculate the second derivatives of the “Bipolar, Non-Vectorial Leads” to calculate the values generated by each Exploring or Negative Electrode and the values generated by the Common or Positive Electrode.

It is a further feature of the present invention to preserve, on a Digital Disk, the electrocardiographic digital data sets, including the subject's identification and the exact anatomical placement of the Exploring or Negative Electrodes used for the electrocardiographic recording.

It is a further feature of the present invention to compare the stored identified electrocardiographic digital data sets with the newly obtained identified electrocardiographic digital data sets.

It is a further feature of the present invention to report and save any differences between the previous and new identified electrocardiographic digital data sets for further evaluations.

### DESCRIPTION OF THE DRAWINGS

This invention and its advances over the prior art can best be understood by reading the specification which follows in conjunction with the drawings herein, in which; according to one embodiment of the present invention:

FIG. 1 is a block diagram of an electrocardiographic method in which the “Common or Positive Terminals” of amplifiers 2001 to 2000+n are connected to an electrode placed on one of the legs of the subject, and the “Exploring or Negative Terminals” are connected to electrodes placed on the cephalic two thirds of the subject's torso.

FIG. 2 is a master flow chart for the microprocessor's different stages.

FIG. 3 is a block diagram of an electrocardiographic method in which the “Common or Positive Terminals” of the amplifiers 2001 to 2000+n and F are connected to a “Constant Value Electrode” and the “Exploring or Negative Terminal” of the amplifiers 2001 to 2000+n are connected to electrodes placed on the cephalic two thirds of the subject's torso and the “Exploring of Negative Electrode” of amplifier F is connected to an electrode placed on the subject's left leg.

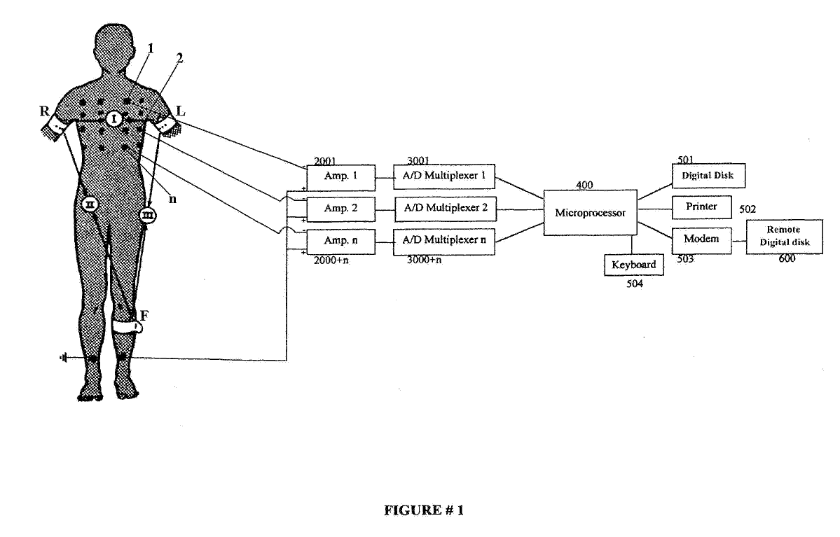

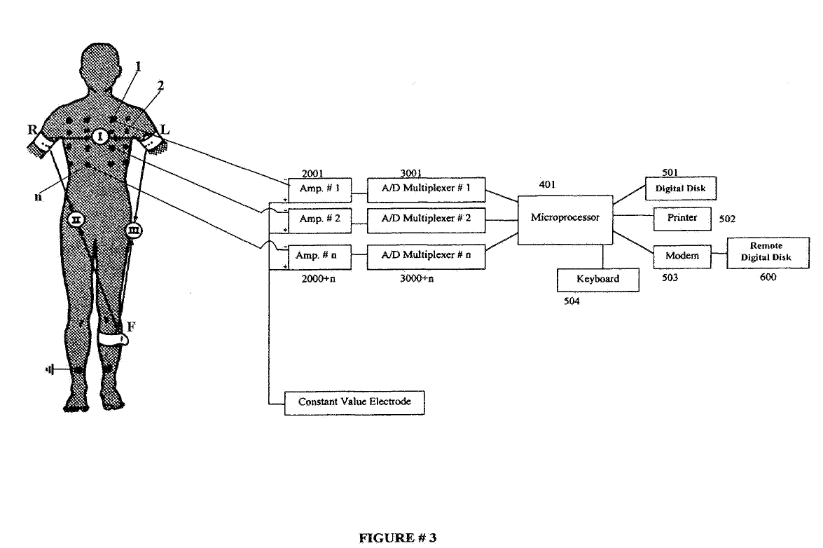

To emphasize the difference between the Bipolar, Non-Vectorial Leads of the present invention and the standard leads of today's electrocardiographic art, the standard Bipolar Vectorial Leads are schematized in FIGS. 1 and 3 on the diagram of the subject.

### DETAILED DESCRIPTION OF THE INVENTION

The embodiments according to the present invention will now be described in detail with reference to the drawings. The different electronic components described in the embodiments; amplifiers, A/D multiplexers, digital filters, calculators, analyzers, digital disks, modems, keyboards, and printers are commercially available components. In the Bipolar, Non-Vectorial Electrocardiography method, the placement of electrodes on a body surface differs significantly from the placement of the electrodes used for over 70 years in today's art electrocardiography.

Approximately twelve Exploring or Negative Electrodes are placed on the subject's cephalic two thirds of the torso according to the areas of prevalence of each component of the myocardium:
1. To obtain electrical potentials generated by an auricle said Exploring or Negative Electrodes are to be placed on the right supra and infra-clavicular areas and on the left pre-sternal or mammary areas.
2. To obtain electrical potentials generated by an antero-lateral surface of a right ventricle the Exploring or Negative Electrodes are to be placed on the anterior surface of the cephalic two-thirds of the right hemi-thorax.
3. To obtain electrical potentials generated by an antero-lateral surface of a left ventricle the Exploring or Negative Electrodes are to be placed on the antero-lateral surface of the caudal two-thirds of the left hemi-thorax.
4. To obtain electrical potentials generated by a postero-inferior surface of said left ventricle the Exploring or Negative Electrodes are to be placed on the posterior surface of the lower half of the left hemi-thorax, left lower back or left leg.

The Exploring or Negative Electrodes are to be identified by their anatomical placement by:
1. the use of easily recognizable anatomical reference points on the anterior and posterior surfaces of the body,
2. by the distance from the midline of the body at the level of the anatomical reference points to the center of the electrode, and
3. the distance between the center of the electrode and the respective right or left medial axillary line.

The anatomical reference points on the anterior surface of the body are:
the supra-sternal notch,
the inter-costal spaces, and
the xiphoid process.

On the posterior surface they are:
the spinal process of the sixth cervical spine, and
the inter-vertebral spaces of T1-T2 to T12-L1.

On the anterior surface electrodes placed above the sternal notch or bellow the xiphoid process two more measurements should be included, they are:
1. The distance from the anatomical reference point to a point where the medial line is transected by a horizontal line that passes though the center of the electrode.
2. The distance from said point in the medial line to the center of the electrode.

The Bipolar, Non-Vectorial Leads are to be analyzed as generated by monophasic electrical potentials present on a surface of the different structures of a myocardium during myocardial systole and diastole and propagated, to specific areas on the surface of the body, through muscular masses (located in the anterior and lateral walls of the chest and abdomen, the diaphragm, and the para-spinal tracks) that are in close contact with them.

FIG. 1 shows an overall view of a modified electrocardiograph as it pertains to the first embodiment of the present invention. As shown, the cephalic two thirds of a torso is connected through the desired number of Exploring or Negative Electrodes n, to a negative terminal of amplifiers 2001 to 2000+n, a left leg is connected through a Common or Positive Electrode to a positive terminal of amplifiers 2001 to 2000+n, to create Bipolar, Non-Vectorial Leads, and a Ground Electrode placed on a right leg is connected to a ground terminal of amplifiers 2001 to 2000+n, to reduce noise. Each high-gain, low-noise, identical amplifier 2001 to 2000+n) has an input isolation switch to prevent current leakage to the subject. The figure, for simplicity, shows only three electrodes placed on the subject's chest and one placed on the distal third of his left leg.

Each amplifier is connected to its own individual Analog-to-Digital multiplexer (3001 to 3000+n). The multiplexer will sample a n amplified analog Bipolar, Non-Vectorial Leads or first derivatives at a rate of around 10,000 samples per second with 12-64-bit resolution to generate n digital data sets, that are fed to a microprocessor (400) connected to the amplifiers (2001 to 2001+n).

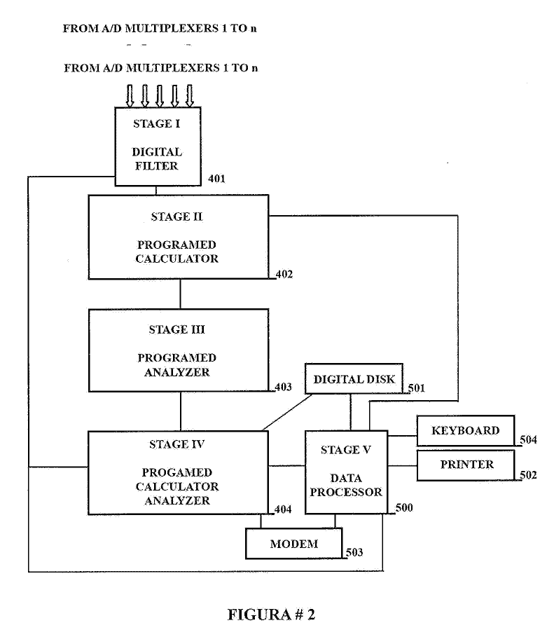

FIG. 2 shows the flow through the Microprocessor's different stages.
1. The first stage is a digital filter (401) with a band-pass filter between 0.5-55 Hz and 65-1000 Hz and band-stop filters between 55-65 Hz and all frequencies below 0.5 Hz and above 1000 Hz. A n filtered digital data is forwarded to a second (402), third (403), and fourth (404) stages of said microprocessor (400) connected to said digital filter (401).
2. Said second stage, comprised of a programmed calculator (402), pairs said filtered digital data sets and subtracts the sets from each other to obtain a digital data set of a calculated or second derivative. Pairing is to be done by subtracting, the left hemi-thorax leads from the right hemi-thorax leads, the cephalic third of the anterior chest leads from the lower two thirds of the peri-sternal leads, and the posterior leads from the sternal leads. Said second derivative leads are fed to said third stage (403) and to a data processor (500) connected to said programmed calculator (402).
3. The third stage comprised of an analyzer (403) compares the n filtered digital data sets of the first derivative with the digital data sets of the second derivative to obtain an approximate values generated by said Common or Positive Electrode placed on the left leg of the subject. Digital data sets of said approximate values generated by the leg electrode are fed to said fourth stage programmed calculator and data analyzer (404) connected to the third stage analyzer (403).
4. The fourth stage, comprised of said programmed calculator and a data analyzer (404), subtracts the digital data set of the approximate values generated by the left leg from the n digital data sets of the first derivative, a difference giving a digital data set of values generated by each individual electrode. All said n digital data sets of the values generated by each electrode and said digital data set of the values of the electrode on the leg, are fed to a data processor (500) connected to the programmed calculator and data analyzer (404).
5. In the fifth stage, comprised of said data processor (500), the operator identifies the n digital data sets of the first derivatives and the n and F digital data sets of the values generated by each individual electrode by the anatomical placement of each Exploring or Negative Electrode and the placement of the Common or Positive Electrode, generating an identified digital data set.
6. If there are no previous electrocardiograms, said identified digital data sets are fed to: a printer (502), connected to the data processor, to print the electrocardiogram, a digital disk (501), connected to the data processor (500), and/or a modem (503), connected to the data processor (500), to save the identified electrocardiographic digital data sets of the subject on a digital disk.
7. If there are previous electrocardiograms, the stored identified electrocardiographic digital data sets of the previous electrocardiograms are retrieved, by said digital disk (501), and fed to the microprocessor's fourth stage programmed calculator and data analyzer (404), connected to the to the digital disk (501), to find if there are differences between the present and prior electrocardiograms.
8. If no changes are found, no new digital data sets are generated.
9. If there are changes, the changes will be reported in new digital data sets that are fed to the Data Processor (500), connected to the programmed calculator and data analyzer (404), to the printer (502), connected to the data processor (500), to be printed, the digital disk (501), connected to the data processor (500) to be stored in a digital disk and to the modem (503), connected to the data processor (500), to be stored in a distant external digital disk (600), connected to the modem (503).
10. If the second derivative digital data sets are needed to make a definitive diagnosis the data processor will send, by request from the operator through the keyboard (504), connected to the data processor (500), the second derivative digital data sets to the printer (502), connected to the data processor (500), to be printed, the digital disk (501), connected to the data processor (500), to be stored in a digital disk and to the modem (503), connected to the data processor (500), to be stored in a remote digital disk (600), connected to the modem (503).

FIG. 3 shows a second embodiment of the present invention. To generate “unipolar” electrocardiograms the subject is positioned so that the cephalic two thirds of the torso and the leg are connected through electrodes to the desired number of “Exploring or Negative Terminals” and the “Common or Positive Terminal” of amplifiers 2001 to 2000+n and F are connected to a “Constant Value Electrode”. The figure is simplified to show only three electrodes: 1, 2 and n.
1. The Exploratory or Negative electrodes are connected to the negative Terminal of each individual high-gain, low-noise, input-switch-insulated amplifier (20001 to 2000+n and F). The positive terminals of the amplifiers (20001 to 2000+n and F) are connected to a “Constant Value Electrode”.
2. The amplified analog electrocardiographic traces are fed to individual analog/digital multiplexers (3001 to 3000+n and F), connected to amplifiers (20001 to 2000+n and F). The multiplexer will sample an n and F amplified analog Bipolar, Non-Vectorial Electrocardiographic lead or first derivative at a rate of around 100,000 samples per second with 12-bit resolution to generate n and F digital data sets that are fed to a Microprocessor (400) connected to the amplifiers (2001 to 2000+n and F).
3. The first stage is a Digital Filter (401) with two-band pass filters between 0.555 Hz and 65-1000 Hz and band stop filters between 55-65 Hz and all frequencies below 0.5 Hz and above 1000 Hz
4. The n filtered digital data sets are forwarded to the fifth stage of the microprocessor comprised of a data processor (405), connected to the Digital Filter (401). The operator identifies the filtered digital data sets by the anatomical localization of the Exploring or Negative Electrodes, the placement of the Common or Positive Electrode, and the subject's identification data. These identified filtered digital data sets are processed according to different commands from the operator.
5. If there are no previous electrocardiograms, the digital data sets are fed to: the printer (502), connected to the data processor (500), to print the electrocardiogram, the disk drive (501), connected to the data processor (500), and/or the modem (503), connected to the data processor (500), to save the identified electrocardiographic digital data sets of the subject on a remote digital disk (600), connected to the modem (503).
6. If the subject has a previous “bipolar” electrocardiogram, the filtered digital data sets 1 to n and F, are fed to the microprocessor's second stage programmed calculator (402) to individually subtract from them the filtered digital data set of the amplifier F to generate “bipolar” Electrocardiograms.
7. These “bipolar” filtered electrocardiograph digital data sets are feed into the next stages of the microprocessor to follow the process described in the previous embodiment.
8. If the previous electrocardiogram was “unipolar”, the identified electrocardiograph digital data sets retrieved from the digital disk by the digital disk (501), connected to the programmed calculator and data analyzer (404), are fed to the microprocessor's fourth stage (404). Said stage's analyzer compares the previous sets of unipolar identified electrocardiograph digital data with the new sets of unipolar identified electrocardiograph digital data.
9. The subsequent stages follow the steps 7, 8, and 9 described in the previous embodiment.

Advantages

Besides the abolition of the erroneous hypothesis accepted in the standard electrocardiogram of today's art, the new “Bipolar Non-Vectorial Electrocardiogram” facilitates the diagnosis of the pathology of the myocardial structure affected as described bellow:
1. Leads from the areas where electrical potentials generated by the contraction of the auricle are prevalent will facilitate the recognition of:
   Arrhythmias of supra-ventricular origin,
   Delays in the A-V conduction, and
   Hypertrophy of the different Auricular chambers;
2. Leads from the areas where electrical potentials generated by the contraction of the right ventricle are prevalent will facilitate the recognition of:
   Arrhythmias originating on the different structures of the Bundle of His in special of the right branch,
   Hypertrophy or enlargement of the right ventricle,
   Angina of the right ventricle, and
   Localization, identification of obstructed artery and extent of the involved area in infarctions of the antero-lateral surface of the right ventricle;
3. Leads from the areas where electrical potentials generated by the contraction of the antero-lateral surface of the left ventricle are prevalent will facilitate the recognition of:
   Arrhythmias originating on the different structures of the Bundle of His in special of the left or of the anterior branches,
   Hypertrophy or enlargement of the left ventricle,
   Angina of the antero-lateral surface of the left ventricle,
   Localization, identification of obstructed artery and extent of the involved area in infarctions of the antero-lateral surface of the left ventricle
   Diagnosis and localization aneurisms of the antero-lateral surface of the left ventricle, and
   Diagnosis and localization of arrhythmias originating in the antero-lateral wall of the left ventricle;
4. Leads from the areas where electrical potentials generated by the contraction of the auricle are prevalent will facilitate the recognition of:
   Arrhythmias originating on the different structures of the Bundle of His in special of the left or of the posterior branches,
   Hypertrophy or enlargement of the left ventricle,
   Angina of the postero-inferior surface of the left ventricle,
   Localization, identification of obstructed artery and extent of the involved area in infarctions of the postero-inferior surface of the left ventricle,
   Diagnosis and localization aneurisms of the postero-inferior surface of the left ventricle, and
   Diagnosis and localization of arrhythmias originating in the postero-inferior wall of the left ventricle.

Since certain changes may be made in the above constructions without departing from the scope of the invention, it is intended that all matter contained in the above description be interpreted as illustrative and not limiting in any way. It is also to be understood that the following claims are to cover all generic and specific features of the invention described herein, and all statements of the scope of the invention which, as a matter of language might be said to fall there between.

### DEFINITION LIST

“Unipolar”, Measurements between terminal pairs when one terminal is connected to a “Constant Value Electrode” and the other is connected to an electrode placed on the subject.

“Bipolar”, Measurements between terminal pairs when both terminals are connected to electrodes placed on the subject.

“Ground Electrode”, Electrical connection to the ground.

“Constant Value Electrode”, Electrode connected to an element of known electrical potential that is constant and free of interference from the electrical fields of the subject and the environment.

“Value”, Electrical potential difference between amplifier terminal pairs.

“Exploring or Negative Terminals”, Negative terminal of the individual amplifiers.

“Common or Positive Terminals”, Positive terminal of the individual amplifiers.

“Exploring or Negative Electrodes”, Electrodes connected to the negative terminal of the amplifiers and placed on the subject's torso.

“Common or Positive Electrode”, Electrode connected to the positive terminal of the amplifiers and placed on the distal third of either leg or right arm.

“Electrocardiographic Lead”, Difference between the electrical pairs of each individual amplifier and identified by the anatomical site of the “Exploring or Negative Electrode” in the subject's torso.

“Digital disk”, Systems used to store digital data. Floppy disk, CD, Hard disk, DVD, etc.

“Bipolar Vectorial Lead”, Today's art Standard Electrocardiographic traces, LI, LII and LIII

“Unipolar Vectorial Lead”, Today's art Wilson's unipolar precardial leads, V1, V2, V3, V4, V5, V6.

“Augmented Unipolar Vectorial Lead”, Today's art Goldberger augmented extremity leads, aVr, aVl, and aVf.

“Bipolar Non-Vectorial Lead”, Leads obtained taking in consideration the new finding that Einthoven's Equilateral Triangle and his Central Dipole do not exist, and the two electrodes are on the body.

“Unipolar Non-Vectorial Lead”. Leads obtained taking in consideration the new finding that Einthoven's Equilateral Triangle and his Central Dipole do not exist, and the negative electrode is on the body and the positive electrode is isolated from the body.

What I claim is:

1. A method of registering Bipolar Non-Vectorial Electrocardiographic leads, comprising of the following steps:
a. placing approximately twelve “Exploring or Negative Electrodes” on a surface of a body were changes of electrical potential are prevalent for each myocardial structure,
b. connecting each of said “Exploring or Negative Electrodes” to a negative terminal of their respective individual amplifier,
c. placing a “Common or Positive Electrode” on a left leg,
d. connecting each said “Common or Positive Electrodes” to a positive terminal of all amplifiers,
e. placing one “Ground Electrode” on a right leg, and
f. connecting said “Ground Electrode” to a ground terminal of all the amplifiers,
whereby said registered Bipolar non-Vectorial electrocardiographic leads will facilitate, understanding normal and pathological physiological processes, and diagnosing normal and pathological processes associated with myocardial systole and diastole.

2. A method of analyzing said Bipolar Non-Vectorial Electrocardiographic Leads, comprising of the following steps:
a. recognizing that a change in electrical potential on said surface of said body synchronized with the contraction of a myocardium is generated by a monophasic electrical potential present on a surface of a structure of said myocardium,
b. recognizing that said change in electrical potential on the surface of the body is propagated throughout through the body by a muscular mass that is in close contact with said structure of the myocardium,
c. recognizing that the structure of the myocardium propagates throughout through the body said monophasic electrical potentials present on said surface of the structure of the myocardium to a specific area of the surface of the body at a specific time, amplitude and morphology,
whereby said analysis facilitates the recognition of the normal processes and facilitates recognition and localization of: abnormal rhythms, alterations of conduction of impulses along the Bundle of His, specific coronary alterations involved in angina and myocardial infarctions, myocardial aneurisms.

3. A method of analyzing a wave of a Bipolar Non-Vectorial Electrocardiographic Lead trace, comprising of the following step:
a. recognizing that said wave of said Bipolar Non-Vectorial Electrocardiographic trace is generated by a difference between specific times, amplitudes and morphologies of monophasic electrical potentials present on a surface of different structures of a myocardium,
whereby said analysis of the waves facilitates the recognition, localization, and myocardial origin of the abnormal Bipolar Non-Vectorial Electrocardiographic Lead.

4. A method of analyzing a segment of a Bipolar non-Vectorial Electrocardiographic trace, comprising of the following step:
a. recognizing that said segment of said Bipolar non-Vectorial Electrocardiographic trace is generated by a difference between specific times, amplitudes and morphologies of the monophasic electrical potentials present on the surface of the different structures of the myocardium,
whereby said analysis of the segments facilitates the recognition and site of ischemic pathology of the myocardium.

5. A method of reporting a Bipolar, Non-Vectorial Electrocardiographic Lead, comprising of the following steps:
a. including with a final printed report a digital disk with a digital data set,
b. burning said digital disk with said digital data sets of all said Bipolar, Non-Vectorial Electrocardiographic Leads acquired by said electrocardiograph,
c. burning the digital disk with the digital data sets of the calculated second derivative leads,
d. burning the digital disk with the digital data sets of a difference between old and new Bipolar, Non-Vectorial Electrocardiographic Leads,
e. burning the digital disc with the digital data sets of the identification of the subject,
f. burning the digital disc with the digital data sets of the anatomical location of the “Exploring or Negative Electrodes”,
whereby said reporting will allow the comparison, by the electrocardiograph, between the present electrocardiographic traces and future traces to facilitate the recognition of incipient pathology or clear pathological process iminating human error
